(19) United States
(12) Patent Application Publication
(10) Pub. No.: US 2008/0034618 A1
(43) Pub. Date: Feb. 14, 2008

(54) Sole device fitting with a foot and method for making the same

(76) Inventor: Li-Yu Lin, Taichung City (TW)

Correspondence Address: RABIN & Berdo, PC, 1101 14TH STREET, NW, SUITE 500, WASHINGTON, DC, 20005, US

(21) Appl. No.: 11/501,115

(22) Filed: Aug. 9, 2006

(51) Int. Cl. A61F 5/14

(52) U.S. Cl. 36/154

(57) ABSTRACT

A sole device includes a bottom layer, a top layer, a check valve and a deformable material. The top layer is made of extendable substance and is attached to a top surface of the bottom layer with its edge being sealed with the bottom layer. A chamber is defined between the two sealed layers. The check valve is mounted in the bottom layer and has a valve inlet. The valve inlet communicates with the chamber to permit filling the deformable material into the chamber through the valve inlet. The deformable material is a viscous fluid and will be cured to an elastic solid by cross-linking after a curing period. The elastic solid cured by the deformable material fits the wearer's foot to provide good comfort and support.

### BACKGROUND

1. Field of Invention

The present invention relates to a sole device for a shoe. More particularly, the present invention relates to a sole device such as an insole or a midsole that has a biomechanical design to fit with the foot to provide comfortable sensation.

2. Description of Related Art

People wear shoes to protect their feet. Generally, the midsoles of the shoes or the insoles for the shoes provide some shock absorption to protect our feet and benefit people with foot comfort. However, since the midsoles or the insoles in accordance with prior art are generally flat, contact points between a sole of the foot and the midsole or insole are imbalanced and distributed mainly in between the toes, the ball of the foot and the heel. The arch between the ball of the feet and the heel is unsupported so that the imbalances easily cause pain and discomfort after a person wears the shoes for a long time.

Some shoe midsoles or insoles (collectively called sole devices) use an arch support inside the shoes to support the arch of the foot to efficiently balance pressure distribution for the foot caused by walking or exercising. However, this kind of sole device with the arch support cannot be completely satisfied because various shapes of the feet. For some people who have flat feet, heel pain, knee pain and shin pain etc., using the conventional arch supports will not alleviate problems caused by the imbalances of the foot. Besides, some patients such as diabetics need to wear shoes that fit exactly with their feet because of their foot care needs. Diabetes decreases peripheral blood circulation and peripheral nerves sensation. The injuries of diabetics are slow to heal. With a diabetic foot, a wound as small as a blister from wearing a shoe can cause a lot of damage. When the wound is not healed, it may be infected, and the infections spread quickly for a diabetic.

Some solutions attempt to directly use foaming material to make the sole devices. The foaming material is softened by heat in an oven, and then the softened sole device is inserted into the shoes. The foot of the wearer who wears the shoes directly deforms the softened sole device to form an insole fitting with the wearer's foot when cooled sufficiently. Although, this method can make an insole that fits with the wearer's foot, this method needs to use thick layer of foaming material. The foaming material needs to be trimmed for some foot parts, especially for the ball of the foot and the heel to accomplish the operation of deforming the foaming material by pressing. Consequently, much foaming material is wasted, which raises manufacturing expense, consumes manufacturing time and is not eco-friendly.

Besides, the sole device for some shoes is required to have large thickness and high hardness because the sole device must provide efficient supporting effect. Thus, the wearer will feel uncomfortable and the foot of the wearer may be injured.

Therefore, there is a need to provide an improved sole device to mitigate or obviate the aforementioned problems.

### SUMMARY

An object of the present invention is to provide a sole device that is ergonomically designed to fit the wearer's foot, and provides comfort and good support to the foot.

Another object of the present invention is to provide a method of making a sole device, and the method is quick and easy, and reduces waste materials during processing.

A sole device in accordance with the present invention comprises a bottom layer, a top layer, a check valve and a deformable material. The bottom later corresponds to the sole of a shoe. The top layer is made of extendable substance and is attached to a top surface of the bottom layer with its edge being sealed with the bottom layer. A chamber is defined between the two sealed layers. The check valve is mounted in the bottom layer and has a valve inlet. The valve inlet communicates with the chamber to permit filling the deformable material into the chamber through the valve inlet. The deformable material is a viscous fluid and will be cured to an elastic solid by a cross-linking reaction after a curing period.

Since the wear treads on the sole device deforms the shape of the deformable material in the chamber, the sole device changes its shape so that the top of the sole device can fit with the wearer's foot before the deformable material is cured. The sole device can be manufactured as an insole or a midsole in the shoe so that the cured deformable material provides good support and comfort to the toes, the ball of the foot and the arch etc.

Consequently, the sole device has some advantages.

First, the sole device uses and seals two layers to form the chamber, and injects the deformable material into the chamber through the check valve. The deformable material in the chamber can flows and changes its shape to fit with the wearer's foot before the deformable material is cured. Therefore, the sole device such as the insole or the midsole can provide a sufficient support and comfort for the toes, the ball of the foot and the arch so that the wear will not feel uncomfortable.

Second, the deformable material in the chamber of the midsole adopts soft and elastic colloid material such as Silicon, which also provides sufficient support and comfort for the foot so that the wearer will not feel the shoe is too hard.

Third, the sole device contains a constant amount of the deformable material to cure so that the sole device eliminates the processes to trim and heat the foamed material. The problem of producing waste material has been addressed. The process for making the sole device is quick and convenient.

### BRIEF DESCRIPTION OF THE DRAWINGS

These and other features, aspects, and advantages of the present invention will become better understood with regard to the following description, appended claims, and accompanying drawings where:

FIG. 1 is an exploded perspective view of a sole device in accordance with the present invention;

FIG. 2 is sectional view of the sole device in FIG. 1;

FIG. 3 is a top plan view of an alternative embodiment of the sole device;

FIG. 4 is sectional view along line 44 of the sole device in FIG. 3;

FIG. 5 is sectional view along line 5-5 of the sole device in FIG. 4;

FIG. 6 is an exploded perspective view of a third embodiment of the sole device in accordance with the present invention;

FIG. 7 is a sectional view of the sole device in FIG. 6;

FIG. 8 is an operational view of a fourth embodiment of the sole device in accordance with the present invention with a mixing unit;

FIG. 9 is a side view in partial section of an injection unit;

FIG. 10 is an operational sectional view of an alternative mixing unit; and

FIG. 11 is a flow chart of a method of making the sole device.

### DESCRIPTION OF THE PREFERRED EMBODIMENTS

Reference will now be made in detail to the present preferred embodiments of the invention, examples of which are illustrated in the accompanying drawings. Wherever possible, the same reference numbers are used in the drawings and the description to refer to the same or like parts.

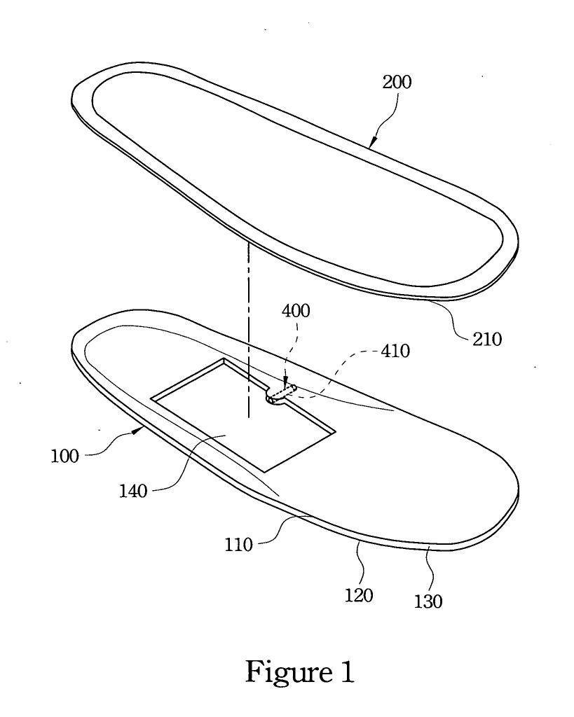

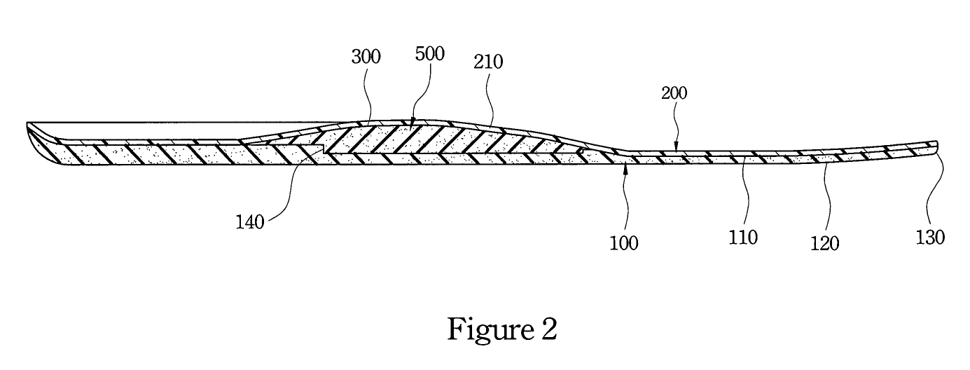

Refer to FIG. 1 and FIG. 2. FIG. 1 and FIG. 2 shows a sole device such as an insole in accordance with the present invention comprising a bottom layer 100, a top layer 200, a check valve 400 and a deformable material 500. The bottom layer 100 has a top surface 110, a bottom surface 120, a side 130 and a recess 140. The recess 140 corresponds to the arch of the foot. The bottom layer 100 may be made of Ethylene-Vinyl Acetate (EVA), Polyurethane (PU), polychloroprene rubber (such as Neoprene), nonwoven fabrics with durable water repellent coating, cardboard with durable water repellent coating (such as Taxon®), or a combination of aforementioned materials.

The top layer 200 is attached to the top surface 110 of the bottom layer 100 with its edge overlapping on the top surface 110 and sealed with the edge of the bottom layer 100. The top layer 200 has a bottom surface 210 facing the bottom layer 100. The top layer 200 is made in the form of a thin film and may be made of extendable and airtight materials, such as Poly Ethylene (PE), Ethylene-Vinyl Acetate (EVA), Polyurethane (PU) or polychloroprene rubber (such as Neoprene). The edges of the two layers 100,200 are connected by adhesive so that the two layers 100,200 compose a body of the insole.

The body of the insole has a chamber 300 between the top layer 200 and the bottom layer 100. The chamber 300 is sealed when the edges of the two layers 100,200 are adhesively connected.

The check valve 400 is mounted in the bottom layer 100 and corresponds to the middle of the shoe. The check valve 400 has a valve inlet 410. The valve inlet 410 allows fluids to be pumped into the chamber 300 in only one direction through the valve inlet 410. In this embodiment, the check valve 400 can use a filling valve disclosed in U.S. Pat. No. 4,129,951 or Taiwan Patent Number 378726. A skilled person in this art can recognize that a description for the check valve 400 is necessary to be provided.

The deformable material 500 is made of polymer substance and may use viscous liquid substance, such as silicone, Polyurethane (PU), Epoxy or any combination of at least two viscous substances. The Polyurethane may be one-component Polyurethane or multi-component Polyurethane. The deformable material 500 is filled into the chamber 300 through the valve inlet 410 of the check valve 400 before the wearer wears the shoe. The deformable material 500 in the chamber 300 is cured to form a soft and elastic colloid solid during a period of curing time, where the curing method for the deformable material 500 is provided later. The deformable material 500 provides good cushioning and supporting effects for the foot.

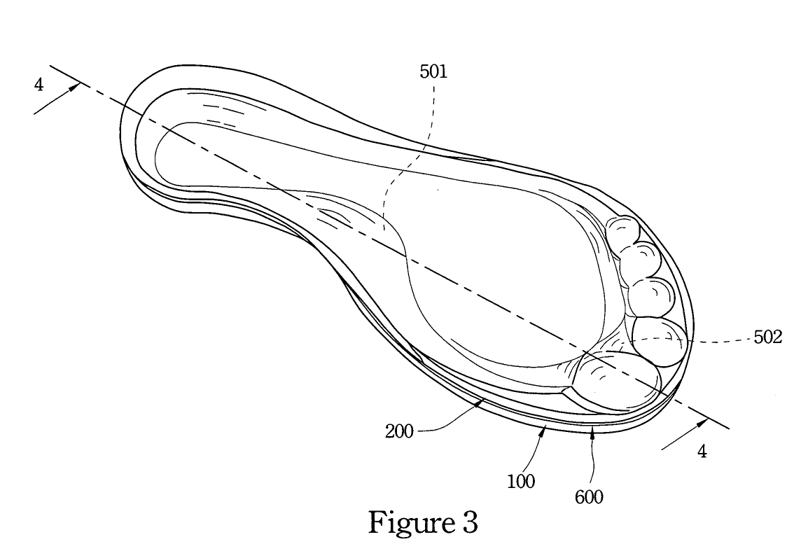

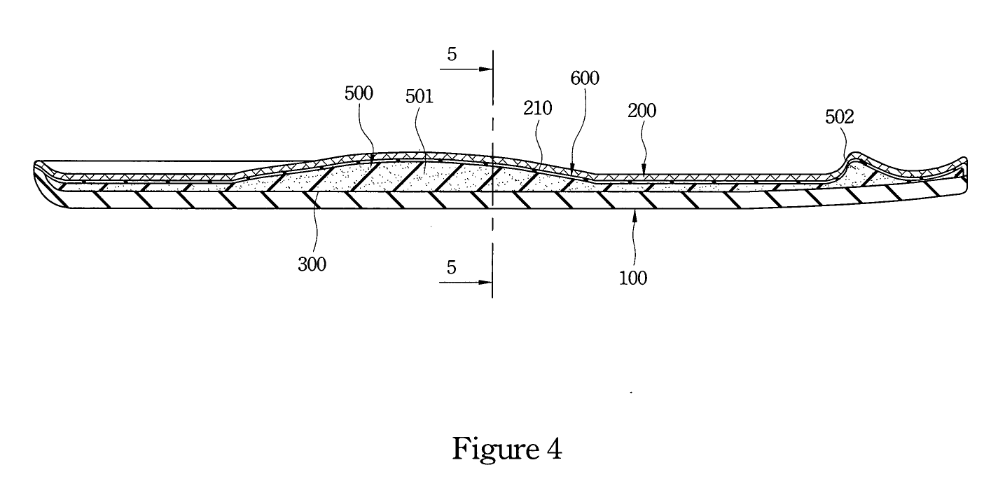

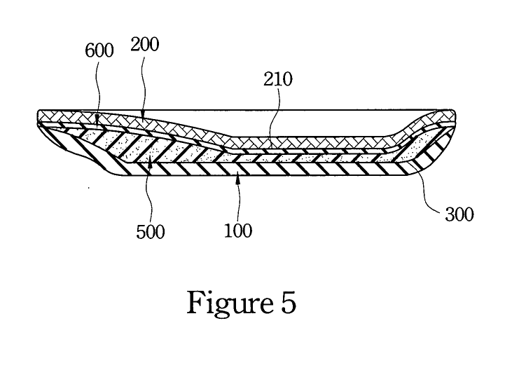

Refer to FIG. 3, FIG. 4 and FIG. 5. A second embodiment in accordance with the present invention has a similar configuration of the first embodiment, but substitutes the material of the top layer 200 with breathable materials and attaches an impervious skin 600 to the bottom surface 210 of the top layer 200. The top layer 200 is made of various artificial or nature fabrics, such as Lycra®, or leather. The top layer 200 is attached to the top surface 110 of the bottom layer 100 to provide comfort for the foot. Furthermore, the top layer 200 may further contain a far infrared composition to radiate far radiation to improve blood circulation and provide thermal insulation for the wearer's foot. The chamber 300 is deformed integrally to a sole shape from the toe area to a heel area.

The impervious skin 600 attached to the bottom surface 210 of the top layer 200 prevents the fluid deformable material 500 from permeating the top layer 200. The impervious skin 600 is made of Thermoplastic Urethane (TPU), Ethylene-Vinyl Acetate (EVA), Polyvinyl acetate (PVA), or waterproof and breathable material.

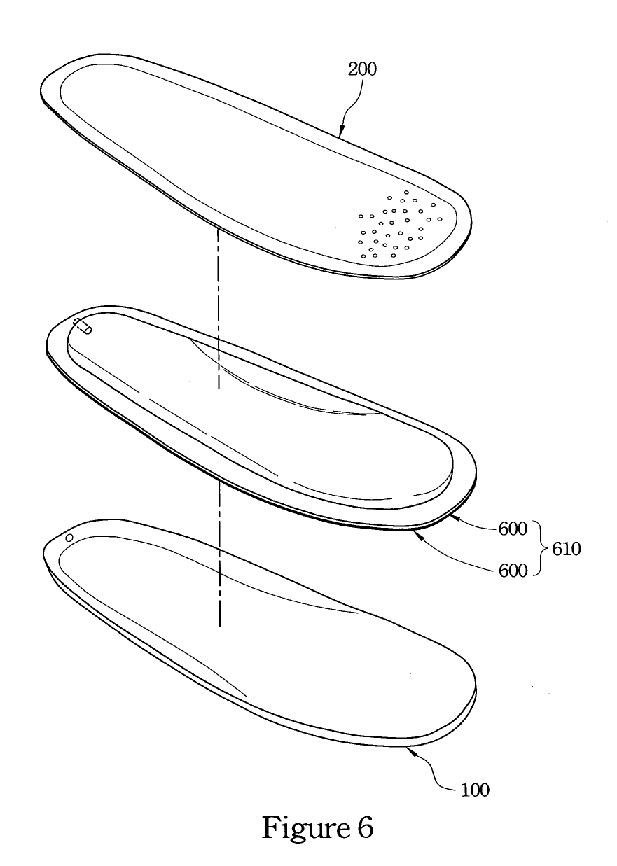

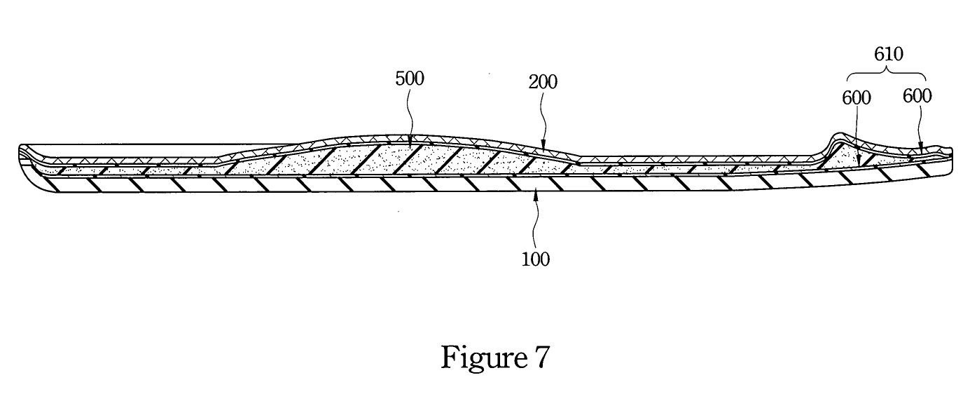

Refer to FIG. 6 and FIG. 7. A third embodiment in accordance with the present invention uses breathable foam materials, such as EVA or PU to make the top layer 200 and the bottom layer 100, and two impervious skins 600. The impervious skins 600 are respectively attached to the bottom surface 210 of the top layer 200 and the top surface 110 of the bottom layer 100. The edges of the impervious skins 600 are connected to each other to form an inner bag 610 that surrounds and holds the deformable material 500. The inner bag 610 is extended to the toe area and the heel area to provide comfort for the whole sole of the foot.

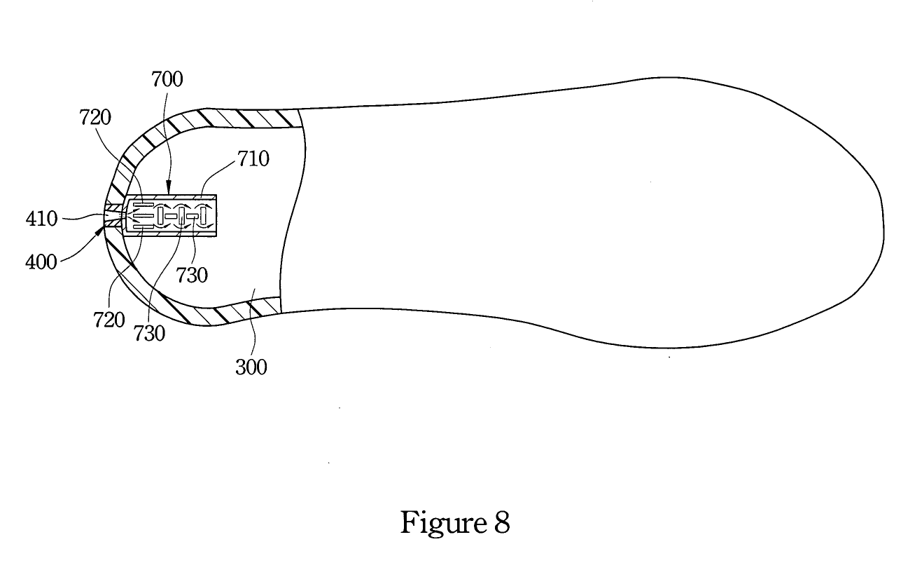

Refer to FIG. 8, a fourth embodiment in accordance with the present invention is implemented with a midsole mounted inside a shoe. The midsole comprises a bottom layer 100, a top layer 200, a check valve 400 and a deformable material 500 as previously described (as shown in FIG. 7), and further comprises a mixing unit 700.

The mixing unit 700 is made of flexible materials such as PE or Polypropylene (PP) and is mounted in the bottom layer 100 prior to the attachment of the two layers 200,100. The mixing unit 700 connects to the valve inlet 410 of the check valve 400 and comprises a casing 710, a plurality of baffles 720 and a plurality of disturbing plates 730. The casing 700 is hollow and connects to the valve inlet 410. The baffles 720 are mounted in the casing 710 and are arranged co-axially with the casing 710. The disturbing plates 730 are mounted in the casing 710 and are arranged in a staggered and parallel manner. The mixing unit 700 mixes different compounds of the deformable material 500 (as shown in FIG. 7) when the deformable material 500 is pumped into the chamber 300. The different compounds of the deformable material 500 are quickly and sufficiently mixed by the mixing unit 700 when the deformable material 500 flows through the mixing unit 700. The baffles 720 and the disturbing plates 730 define multiple and circuitous flow paths (as indicated by arrows in FIG. 8) for the deformable material 500 to disturb and mix the compounds of the deformable material 500.

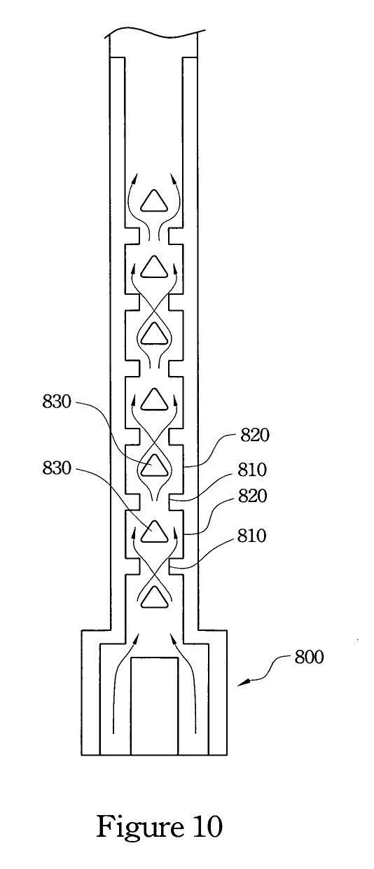

Refer to FIG. 10. FIG. 10 discloses a fifth embodiment in accordance with the present invention with an alternative mixing unit 800. The mixing unit 800 connects the outer side of the check valve to the outside of the midsole and comprises two thin films. The thin films are made of plastic and connected to each other by high frequency soldering. The mixing unit 800 has multiple segments 810, multiple disturbing sections 820 and multiple baffles 830. Each segment 810 has one disturbing section 820 with one baffle 830 inside. The baffle 830 may have a triangular shape. Likewise, the mixing unit 800 also quickly disturbs and mixes the compounds of the deformable material 500 when the deformable material 500 flows through the mixing unit 800. However, the mixing unit 800 of the fifth embodiment is used for mixing multi-component deformable materials, and is unnecessary for mixing one-component deformable materials.

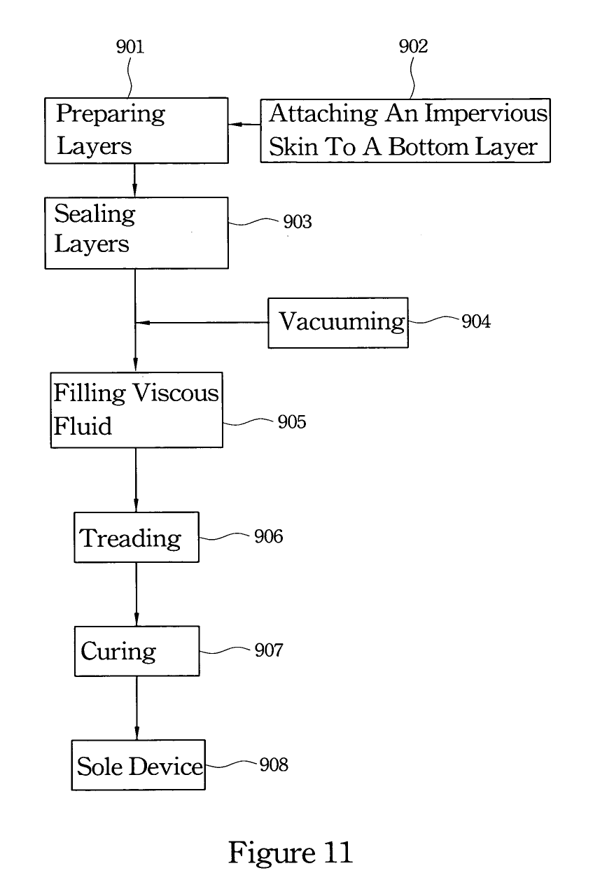

Refer to FIG. 4 and FIG. 11. FIG. 11 discloses a method for making the sole device in accordance with the present invention. Step 901 and step 902 show providing a top layer 200, a bottom layer 100 and a check valve 400 (as shown in FIG. 1) and attaching an impervious skin 600 to the bottom surface 210 of the top layer 200. Then, the edges of the top layer 200 and the bottom layer 100 are sealed by adhesive or high frequency soldering to form a chamber 300 where the chamber 300 communicating with an valve inlet 410 of the check valve 400 as shown in step 903.

The chamber 300 is vacuumed through the valve inlet 410 of the check valve 400 as shown in step 904, so that the chamber 300 stays at negative pressure state to facilitate filling the fluid deformable material. Since air in the chamber 300 has been completely vacuumed out, bubbles will not be produced in the deformable material during the filling operation.

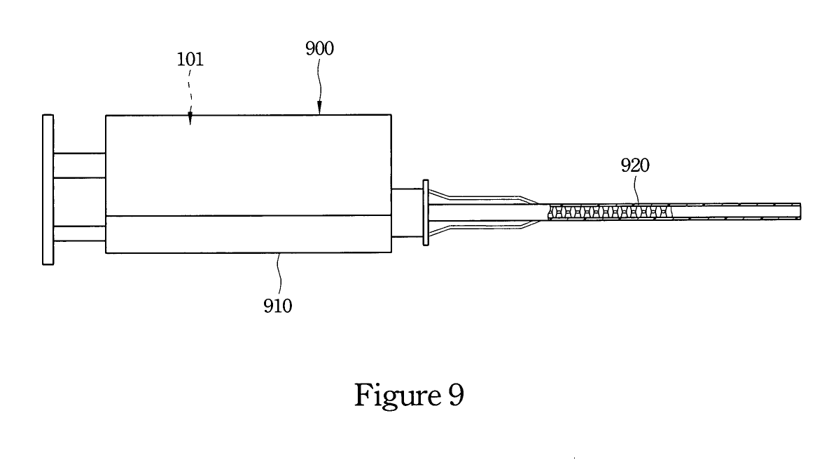

Refer to FIG. 4, FIG. 9 and FIG. 11, the filling operation of the deformable material can use an injection unit 900. The injection unit 900 comprises a body 910 and a nozzle 920 with a spiral propelling device. The body 910 holds the viscous fluid 101 that is injected into the chamber 300 through the valve inlet 410 of the check valve 400 as shown at step 905. The viscous fluid 101 may be two-component silicon. The injection unit 900 may be a dual-action injection cylinder where the nozzle 920 is attached to its head. The nozzle 920 mixes the silicon before the silicon is filled into the chamber 300, or the silicon is mixed by the mixing unit 800 in the chamber 300 as shown in FIG. 8.

The next step is inserting the sole device into a shoe before the curing time of the viscous fluid 101. Thus, a wearer wears the shoe and treads on the sole device in the shoe to deform the viscous fluid 101 in the chamber 300 at step 906 and waits for a curing time of 2 to 5 minutes for curing the viscous fluid 101. The viscous fluid 101 is cured by its internal cross-linking reaction when the components of the viscous fluid 101 are mixed at step 907 and step 908. The viscous fluid 101 is transformed into the form of deformable material 500 that is held in the chamber 300 and is hardened to form an elastic colloid substance after the viscous fluid 101 has cured. The sole device has a protruded arch area 501 and a toe area 502 as shown in FIG. 3 and FIG. 4 to provide sufficient support and comfort for the foot because the top of the sole device has a shape fitted with the wearer's foot in step 908.

Furthermore, an impervious skin 600 can be attached to the top surface of the bottom layer 100 in advance before the two layers 100,200 are combined. The impervious skin 600 can be previously positioned on an upper mold of a set of mold for making the bottom layer 100, and integrally combined with the bottom layer 100 during molding the bottom layer 100. Removing the set of mold obtains one bottom layer 100 with one impervious skin 600 on its top surface. Besides, for different compounds of materials, changing the diameter of the body 910 of the injection unit 900 can obtain an appropriate ratio of the compound of the deformable material to have an optimal foaming and cross-linking effect.

Consequently, the sole device has some advantages.

First, the sole device uses and seals the two layers 200,100 to form the chamber 300, and injects the deformable material 500 into the chamber 300 through the check valve 400. The deformable material 500 in the chamber 300 can flow and change its shape to fit with the wearer's foot before the deformable material 500 is cured. Therefore, the sole device such as the insole or the midsole can provide a sufficient support and comfort for the toes, the ball of the foot and the arch so that the wear will not feel uncomfortable.

Second, the deformable material 500 in the chamber 300 of the midsole adopts soft and elastic colloid material such as Silicon, which also provides a sufficient support and comfort for the foot so that the wearer will not feel the shoe is too hard.

Third, the sole device contains a constant amount of the deformable material 500 to cure so that the sole device eliminates the processes to trim and heat the foamed material. The problem of producing waste material has been addressed. The process for making the sole device is quick and convenient.

It will be apparent to those skilled in the art that various modifications and variations can be made to the structure of the present invention without departing from the scope or spirit of the invention. In view of the foregoing, it is intended that the present invention cover modifications and variations of this invention provided they fall within the scope of the following claims and their equivalents.

What is claimed is:

1. A sole device fitting with a foot, and the sole device comprising
a bottom layer;
a top layer made of extensible material attached to the bottom layer with its edge sealed with the bottom layer where a chamber is formed between the top layer and a bottom layer by sealing the top layer with the bottom layer;
a check valve mounted in the bottom layer and communicated with the chamber and having a valve inlet; and
a deformable material selected from viscous fluid materials and injected into the chamber through the valve inlet of the check valve and held in the chamber where the deformable material is cured by cross-linking to form an elastic colloid substance after a pre-determined period.

2. The sole device as claimed in claim 1, further comprising a mixing unit connected to the valve inlet of the check valve.

3. The sole device as claimed in claim 2, wherein the mixing unit comprises
a hollow casing;
a plurality of baffles mounted in the hollow casing and arranged co-axially with the hollow casing; and
a plurality of disturbing plates mounted in hollow the casing and arranged in a staggered and parallel manner with the plurality of baffles.

4. The sole device as claimed in claim 2, wherein the mixing unit comprises
multiple segments;
multiple disturbing sections formed respectively in the multiple segments; and
a baffle formed co-axially and centrally in each of the segments.

5. The sole device as claimed in claim 1, wherein the deformable material is selected from the group consisting of silicone, Polyurethane (PU), and Epoxy.

6. The sole device as claimed in claim 1, wherein the bottom layer is made by a material selected from the group consisting of Ethylene-Vinyl Acetate (EVA), Polyurethane (PU), polychloroprene rubber, nonwoven fabrics with durable water repellent coating, and cardboard with durable water repellent coating.

7. The sole device as claimed in claim 1, further comprising an impervious skin attached to a bottom surface of the top layer.

8. The sole device as claimed in claim 1, further comprising an impervious skin attached to a top surface of the bottom layer.

9. The sole device as claimed in claim 7, the impervious skin is made by a material selected from the group consisting of Thermoplastic Urethane (TPU), Ethylene-Vinyl Acetate (EVA), Polyvinyl acetate (PVA), and waterproof and breathable material.

10. The sole device as claimed in claim 8, the impervious skin is made by a material selected from the group consisting of Thermoplastic Urethane (TPU), Ethylene-Vinyl Acetate (EVA), Polyvinyl acetate (PVA), and waterproof and breathable material.

11. The sole device as claimed in claim 1, wherein the chamber is deformed integrally to form a sole shape from a toe area to a heel area.

12. The sole device as claimed in claim 1, wherein the top layer and the bottom layer are combined to form an insole for a shoe.

13. The sole device as claimed in claim 1, wherein the top layer and the bottom layer are combined to form a midsole for a shoe.

14. The sole device as claimed in claim 1, the bottom layer further has a recess corresponding to the arch of the foot.

15. A method of making a sole device fitting with a foot, and the method comprising,
(a) preparing a top layer, a bottom layer and a check valve mounted in the bottom layer;
(b) sealing the top layer with the bottom layer, forming a chamber between the top layer and the bottom layer, and communicating a valve inlet of the check valve to the chamber;
(c) filling a viscous fluid into the chamber through the valve inlet by an injection unit;
(d) treading on the top layer by the foot to deform the deformable material in the chamber; and
(e) curing the deformable material in the chamber to form the sole device fitting with the foot.

16. The method as claimed in claim 15, further comprising
attaching an impervious skin to a top surface of the bottom layer before step (a).

17. The method as claimed in claim 15, further comprising
attaching an impervious skin to a bottom surface of the top layer before step (a).

18. The method as claimed in claim 15, further comprising
vacuuming the chamber before step (c).
